(19) United States
(12) Patent Application Publication
(10) Pub. No.: US 2010/0016808 A1
(43) Pub. Date: Jan. 21, 2010

(54) Thin-Walled Delivery System

(75) Inventor: Christopher J. Groppi, Franklin, WI (US)

Correspondence Address: SWANSON & BRATSCHUN, L.L.C., 8210 SOUTHPARK TERRACE, LITTLETON, CO, 80120, US

(73) Assignee: BioForm Medical, Inc.

(21) Appl. No.: 12/175,164

(22) Filed: Jul. 17, 2008

(51) Int. Cl. A61M 5/34

(52) U.S. Cl. 604/243

(57) ABSTRACT

A system for tissue augmentation using a thin-walled needle. The system includes a thin-walled needle and a syringe. The system further includes a plurality of particles for injection into a desired tissue to be augmented. The thin-walled needle has an inner diameter sufficient to allow for passage of the particles and an outer diameter designed to minimize the puncture wound in the skin when the needle is inserted. The particles may be suspended in a carrier.

### BACKGROUND OF THE INVENTION

The present invention relates generally to the field of tissue augmentation systems. Prior art needles typically utilize established dimensions. Generally these prior art needles have been designed for injecting fluids. Typically the size of the puncture by the needle is not of concern.

### SUMMARY OF THE INVENTION

One embodiment of the invention relates to a system for augmenting tissue comprising a needle assembly and tissue augmentation material. The needle assembly includes a hub and a needle. The hub has a first end for engaging the syringe and a second end for engaging the needle. The needle comprises a hallow shaft with a first end and a second end, each end having an opening respectively into an interior volume of the hallow shaft, the first end engaging the second end of the hub and a second end having a pointed tip. The hallow shaft having an outer surface and an inner surface, with a thickness of material there between, the outer surface having a nominal diameter of about 0.353 mm to about 0.367 mm and the inner surface having a nominal diameter of about 0.198 mm to about 0.244 mm, with the nominal thickness of material being about 0.062 to about 0.078 mm. The tissue augmentation material may, in one embodiment, comprise particles suspended in a carrier, wherein the material may be positioned in the syringe. Optionally, the tissue augmentation material can be included in an enclosure. In a further embodiment, the material is transferred to a syringe from a container immediately prior to administration to a patient.

In another embodiment, the invention relates to a kit for augmenting tissue. The kit includes an enclosure having tissue augmentation material disposed therein, and a hub and needle attached to the syringe. The hub has a first end for engaging the syringe and a second end for engaging a needle. The needle comprises a hallow shaft with a first end and a second end, each end having an opening respectively into an interior volume of the hallow shaft, the first end engaging the second end of the hub and a second end having a lancet. The hallow shaft has an outer diameter and an inner diameter, with a thickness of material there between, the outer surface having a nominal diameter of about 0.353 mm to about 0.367 mm and the inner surface having a nominal diameter of about 0.198 mm to about 0.244 mm, with the nominal thickness of material being about 0.062 to about 0.078 mm. The tissue augmentation material is injected into a tissue to be augmented via the needle.

In yet another embodiment, the invention relates to a system for augmenting tissue comprising a needle assembly having a hub and a needle. The hub has a first end for engaging a syringe and a second end for engaging a needle. The needle comprises a hallow shaft with a first end and a second end, each end having an opening respectively into an interior volume of the hallow shaft, the first end engaging the second end of the hub and a second end having a lancet. The hallow shaft has an outer diameter and an inner diameter, with a thickness of material there between, the nominal outer diameter being about 0.0.367 mm and the nominal inner diameter of about 0.0.244 mm, with the nominal thickness of material being about 0.078 mm. A plurality of injectable particles are suspended in a carrier and disposed in the syringe, the plurality of particles having a size distribution within the range of 15 microns to about 65 microns.

In one embodiment, the outer surface of the needle has a nominal diameter of about 0.353 mm (about that of a 28 gauge needle) and the inner surface having a nominal diameter of about 0.198 mm (about that of a 27 gauge needle). In another embodiment, the outer surface of the needle has a nominal diameter of about 0.305 mm (about that of a 30 gauge needle) and the inner surface having a nominal diameter of about 0.198 mm (about that of a 27 gauge needle). In another embodiment, the outer surface of the needle has a nominal diameter of about 0.406 mm (about that of a 27 gauge needle) and the inner surface having a nominal diameter of about 0.254 mm (about that of a 25 gauge needle).

The invention includes certain features and combinations of parts hereinafter fully described, illustrated in the accompanying figures, described below, and particularly pointed out in the appended claims, it being understood that various changes in the details may be made without departing from the spirit, or sacrificing any of the advantages of the present invention.

### BRIEF DESCRIPTION OF THE DRAWINGS

FIG. 1a is an illustration of one embodiment of a tissue augmentation system; FIG. 1b is an closeup of the augmentation material contained in the syringe of FIG. 1a;

FIG. 2 is an illustration of the needle assembly and syringe of FIG. 1;

FIG. 3a is a partial perspective view of one embodiment of a needle; FIG. 3b is a cross-sectional view of the needle of FIG. 3a along line A-A; and

FIG. 4 illustrates a kit containing a needle assembly, a syringe, and a container, the container filled with augmentation material.

### DETAILED DESCRIPTION OF THE PREFERRED EMBODIMENTS

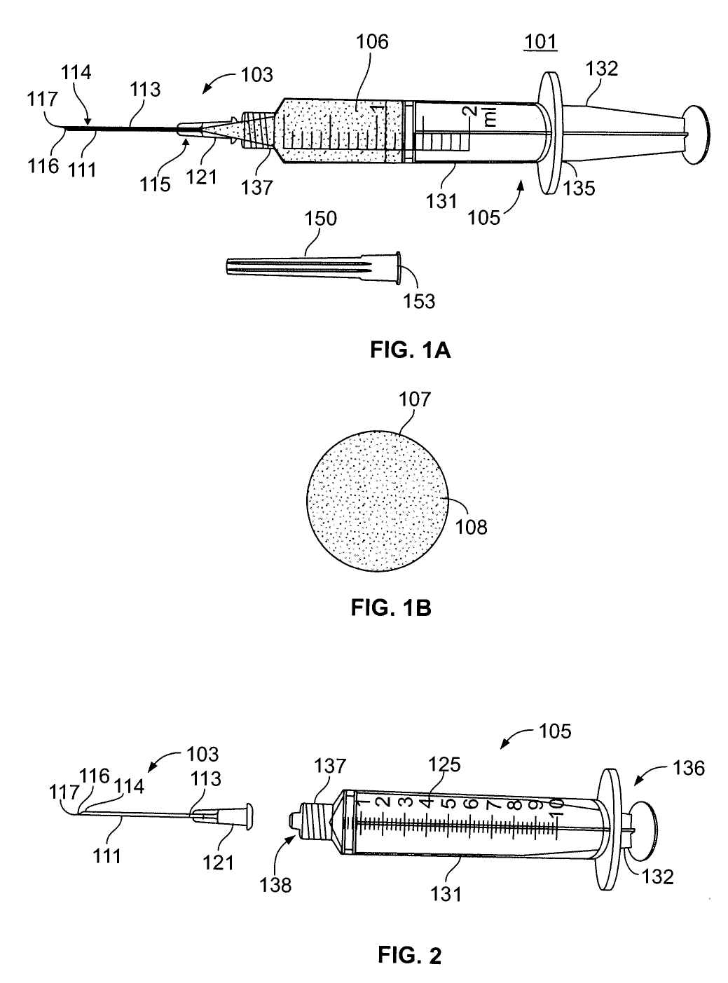

The present invention relates to a needle 111 for use in delivery of augmentation material 106 and kits for providing same. The kit includes a needle 111 as described below and augmentation material 106. In one embodiment, as FIG. 1a illustrates, a delivery system 101 is provided. The delivery system 101 includes a needle assembly 103 and a syringe 105 and augmentation material 106. The needle assembly 103 and syringe 105 are in fluid communication such that, in general, the syringe 105 serves to contain the material 106 (best shown in FIG. 1b), which is ejected from the syringe 105 through the needle assembly 103 and injected into a target tissue to be augmented.

In various embodiments, the augmentation material 106 comprises a plurality of small particles 107. Various such particles 107 are known in the art. Particles 107 used in the delivery system 101 may be formed from “non-biodegradable”, “biodegradable” (by the body) materials or combinations thereof. The particles 107 may include, but are not limited to, silicone gel, Teflon paste, bioplastics including polymerized silicone particles dispersed in polyvinylpyrrolidone, carbon-coated substrate particles comprised of metallic cores, glass, ceramics, microspheres comprising acrylic polymers, acrylate polymers including polymethacrylate, polymethylmethacrylate, poly-L-lactic acid, sodium acrylate polymer, acrylamide polymer, acrylamide derivative polymer or copolymer, sodium acrylate and vinyl alcohol polymers, isobutylene-maleic anhydride crosslinked copolymer, starch-acrylonitrile graft copolymer, crosslinked sodium polyacrylate polymer, crosslinked polyethylene oxide, polylactides such as polylactic acides, polyglycolides, or copolymers thereof and polysaccharides including cellulose such as carboxymethylcellulose, ethylcellulose, hydroxylpropyl cellulose, and hyaluronic acid, alginates, chitosan, gelatin, and silicones, hydrogels, glass and the like.

In an exemplary embodiment, the particles 107 are ceramic based composites. Particulate ceramic materials that can be used to form the particles 107 include, but are not limited to, calcium hydroxyapatite, and other suitable materials including, but are not limited to, calcium phosphate-based materials, alumina-based materials and the like. Examples include, but are not limited to, tetracalcium phosphate, calcium pyrophosphate, tricalcium phosphate, octacalcium phosphate, calcium fluorapatite, calcium carbonate apatite, and combinations thereof. In one embodiment, the ceramic particles are smooth, rounded, substantially spherical, particles of a ceramic material embedded in a biocompatible gel material that is continuous, cross linked or in a dehydrated configuration as discussed below.

In one embodiment, the particles 107, such as those embodiments described above, are suspended in a “carrier” 108. In an exemplary embodiment, the carrier 108 supports the particles 107, facilitating injection through the needle assembly 103. In one embodiment, the carrier 108 forms an integral and compatible part, along with the particles 107, of the implant (and surrounding bioenvironment) once injected. The carrier 108 may be non-biodegradable, biodegradable or a combination thereof. In one embodiment, the carrier 108 may be water. The carrier 108 may further include additives such as collagen.

In one embodiment, the carrier 108 comprises a gel. In a further embodiment, the gel of the present invention exhibits characteristics that modifiable to mimic the physical, chemical and properties of the implant location. Such characteristics include, but are not limited to, extrusion, rheological physical/mechanical parameters, decomposition rate (chemical and physical), moldability, mechanical performance and porosity to modulate tissue response. Gel characteristics control varying rates of resorption, as host tissue forms around the slower resorbing ceramic particles.

The carrier 108 comprises, in one exemplary embodiment, a polymer gel. In one embodiment, the gel is a polysaccharide gel. Polysaccharides that may be utilized in the present invention include, for example, any suitable polysaccharide within the following classes of polysaccharides: celluloses/starch, chitin and chitosan, hyaluronic acid, hydrophobe modified systems, alginates, carrageenans, agar, agarose, oligosaccharide and macrocyclic systems. Examples of polysaccharides grouped into four basic categories include: 1. nonionic polysaccharides, including cellulose derivatives, starch, guar, chitin, agarose and. dextron; 2. anionic polysaccharides including cellulose derivatives starch derivatives, carrageenan, alginic acid, carboxymethyl chitin/chitosan, hyaluronic acid and xanthan; 3. cationic polysaccharides, including cellulose derivatives, starch derivatives guar derivatives, chitosan and chitosan derivatives (including chitosan lactate); and 4. hydrophobe modified polysaccharides including cellulose derivatives and alpha-emulsan. Preferred polysaccharides for use in the present invention include, for example, carboxymethylcellulose, agar methylcellulose, hydroxypropyl methylcellulose, ethylcellulose, microcrystalline cellulose, oxidized cellulose, chitin, chitosan, alginic acid, sodium alginate, and xanthan gum.

Various embodiments of the carrier 108 comprise a gel having crosslinkable components. In such embodiments, appropriate gel cross linkers may, include, but are not limited to: heat, pH, cross-linking through mono valent, di-valent and tri-valent cationic interactions. The cross linking ions used to crosslink the polymers may be anions or cations depending on whether the polymer is anionically or cationically cross linkable. Appropriate cross linking ions include but are not limited to cations selected from the group consisting of calcium, magnesium, barium, strontium, boron, beryllium, aluminum, iron, copper, cobalt, and silver ions. Anions may be selected from but are not limited to the group consisting of phosphate, citrate, borate, carbonate, maleate, adipate and oxalate ions. More broadly, the anions are derived from polybasic organic or inorganic acids. Preferred cross linking cations are calcium iron and barium ions. The most preferred cross linking cations are calcium and iron. The preferred cross linking anions are phosphate, citrate and carbonate. Cross linking may be carried out by contacting the polymers with an aqueous solution containing dissolved ions. Additionally, cross-linking could be accomplished through organic chemical modification including: poly-functional epoxy compound is selected from the group consisting of 1,4-butanediol diglycidyl ether (BDDE), ethylene glycol diglycidyl ether (EGDGE), 1,6-hexanediol diglycigyl ether, polyethylene glycol diglycidyl ether, polypropylene glycol diglycidyl ether, polytetramethylene glycol digylcidyl ether, neopentyl glycol digylcidyl ether, polyglycerol polyglycidyl ether, diglycerol polyglycidyl ether, glycerol polyglycidyl ether, tri-methylolpropane polyglycidyl ether, pentaerythritol polyglycidyl ether, and sorbitol polyglycidyl ether. Additionally, cross-linking could be accomplished through organic chemical modification through the carbonyl or hydroxide functionality of the polysaccharide backbone reaction.

In one embodiment, the gel is carboxymethylcellulose (“CMC”) based with concentration ranges from 0.1% to 10%, preferably from 1.5% to 5% band most preferably from 2% to 3%. Material 106 may be mixed to create composite gels with compositional ranges for each component between 0.1% to 5%. Glycerin or the like or other space occupying filler (including ionic components and other organic/inorganic non reactive components) may be added to the composition and range from 0.1% to 5%.

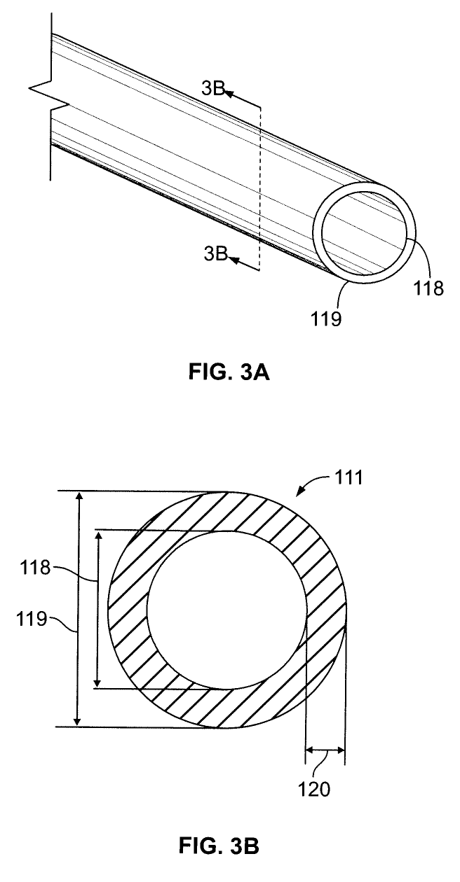

The particles 107, such as those discussed above, alone, or in combination with a carrier 108, as discussed previously, are injected via a syringe 105 and needle assembly 103. FIGS. 1-3 illustrates the needle assembly 103 includes a needle 111 (shown in detail in FIG. 3) and a hub 121. The needle 111 comprises a hallow shaft 113 having a first end 114 through which material 106 exits the needle (i.e., positioned distal the hub 121) and a second end 115 for engaging the hub 121 (i.e., proximate the hub 121). The first end 114 may include a bevel 116. The bevel 116 is a tapered portion of the shaft 113 forming a point 117. In one embodiment, the shaft 113 includes more than one bevel 116, the first to create a taper to slim a diameter of the needle proximate the first end 114 and the second to create the point 117 for piercing the tissue.

The hallow shaft 113 includes an inner diameter 118 and an outer diameter 119 with a thickness 120 there between. The inner diameter 118 provides a fluid flow path for the material 106 to pass through the needle 111. It will be appreciated that smaller inner diameters will result in a more restrictive flow of fluid and will limit the type of materials extrudable from the needle 111, for example suspended particles 107 above a certain size.

The outer diameter 119 of the needle 111 reflects the size of the hole the needle 111 will puncture in the tissue. The larger the outer diameter 119, the larger the puncture in the tissue.

In one embodiment, the outer diameter 119 is minimized while the inner diameter 118 is maximized. In this embodiment, the thickness 120 is sufficient to impart structural support to the needle 111. Thus, the thickness 120 must be such that the needle 111 can support its own weight and that of material 106 being extruded therefrom.

In one embodiment, the needle 111 has an outer diameter 119 that is sized to approximately equal the outer diameter of a first standard needle gauge while the inner diameter 118 is sized to approximately equal the inner diameter 118 of second, larger, standard needle gauge. In another exemplary embodiment, the outer surface having a nominal diameter of about 0.353 mm to about 0.367 mm and the inner surface having a nominal diameter of about 0.198 mm to about 0.244 mm, with the nominal thickness of material 106 being about 0.062 to about 0.078 mm. In one embodiment, the outer surface of the needle has a nominal diameter of about 0.353 mm (about that of a 28 gauge needle) and the inner surface having a nominal diameter of about 0.198 mm (about that of a 27 gauge needle). In another embodiment, the outer surface of the needle has a nominal diameter of about 0.305 mm (about that of a 30 gauge needle) and the inner surface having a nominal diameter of about 0.198 mm (about that of a 27 gauge needle). In another embodiment, the outer surface of the needle has a nominal diameter of about 0.406 mm (about that of a 27 gauge needle) and the inner surface having a nominal diameter of about 0.254 mm (about that of a 25 gauge needle). In one embodiment, the outer diameter of the needle 111 may be selected from the above range and a desired thickness of the needle selected, the thickness structurally sufficient for an intended use, and the inner diameter determined from those two selected measurements.

In one embodiment, particles 107 may range in size about 15 microns to about 65 microns. Preferably from about 15 microns to about 55 microns, more preferably from about 15 microns to about 50 microns, and most preferably about 25 microns to about 45 microns. Concentration of ceramic particles 107 ranges from 5% to 65%, preferably from 10% to 50% and most preferably from 30% to 45%.

In one embodiment, the needle 111 is affixed to the hub 121 with an adhesive, such as but not limited to epoxy.

The syringe 105 includes a body 131 and a plunger 132. The body 131 further includes a body 131, a plunger opening 135 at a first end 136 and a needle connection mechanism 137, with a passage from the body 131 therethrough, at second end 138. The plunger 132 forms a seal with the inner surface of the body 131 such that movement of the plunger 132 into the body 131 will force the contents through the second end 138. The needle assembly 103 connects with the second end 138 via mechanisms known in the art so as to place the needle assembly 103 in fluid communication with the chamber of the body 131. In exemplary embodiments the mechanisms for connecting the needle assembly 103 and the syringe 105 include, but are not limited to, luer locks, threads, and “snap-fit” mechanisms.

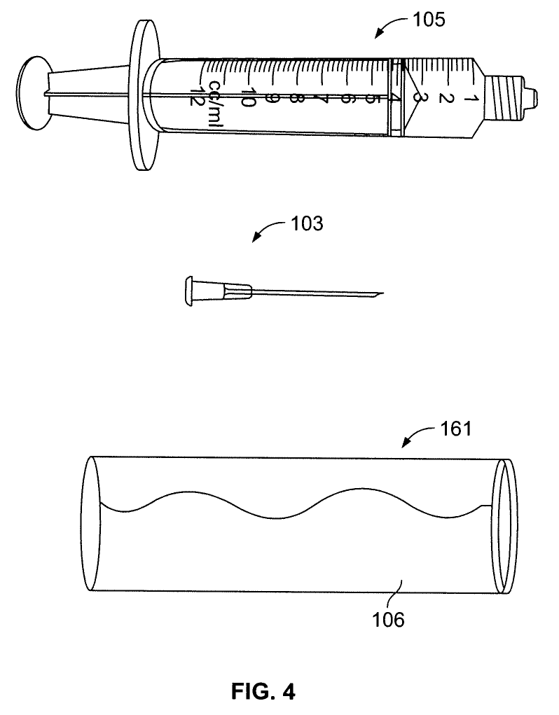

In an exemplary embodiment, a kit (one embodiment shown in FIG. 4) is provided containing the needle and an enclosure 160 having a volume of augmentation material 106. In one embodiment the kit includes sufficient augmentation materials 106 for at least one usage for a give application, i.e., a “single application amount”. In one embodiment, the volume of particles 107 included in the kit varies depending on the intended application. In various embodiments, applications include, but are not limited to: providing tissue implant product throughout the body 131, such as, for example, urinary tract, vocal fold, lip tissue, cheek, other dermal tissue for various uses including clinical and restorative applications and cosmetic applications like augmenting nasolabial folds, nasolabial crease, marionette lines, lip augmentation and augmenting skin wrinkles and folds. FIG. 1 illustrates one such kit.

In one embodiment, the kit may include a one or more needles 111 in combination with the augmentation materials 106. It should be appreciated that the number of needles 111 provided with the kit can be varied as needed, such as providing two needles with a single syringe 105 and augmentation material 106 to provide a second needle in the event the first needle becomes contaminated. Likewise, in one embodiment the kit may include a needle 111 for each single application amount included in the kit, either in separate syringes 105 or containers 161 as described further below.

The material 106 may be pre-packaged into the syringe 105 in the kit. Alternatively, the material 106 may be provided in a container 161 as part of the kit, with an empty syringe 105 provided as well. In a further alternative embodiment, the material 106 may be provided pre-packed into a syringe 105 with additional material 106 in a container 161 or pre-packaged into additional syringes 105. In one embodiment, as discussed above, the amount of material 106 provided in the pre-packed syringe 105 or a container 161 is sufficient for a single application. Alternatively, the material 106 provided in a container 161 with the kit may include sufficient material 106 for multiple applications.

In one embodiment, shown in FIG. 1, a sheath may be provided for covering the needle. The sheath 150 serves to maintain sterility of the need and to provide protection against physical damage of the needle or injury to a person handling the needle assembly 103. The sheath 150 comprises a hollow body 151 slightly longer than the length of the needle, so that the needle 111 can be completely disposed within the sheath 150. The sheath 150 further includes a retention mechanism 153 for retaining the sheath 150 on the needle assembly 103. Such retention mechanism 153 may include engaging the hub 121 by, but not limited to, “snap-fit”, threads, latches, and “friction fit” mechanisms.

Certain embodiments of the syringe 105 may include indicia 125 for providing a user with an indication of the volume of particles 107 present in the syringe.

The foregoing description of embodiments of the present invention have been presented for purposes of illustration and description. It is not intended to be exhaustive or to limit the present invention to the precise form disclosed, and modifications and variations are possible in light of the above teachings or may be acquired from practice of the present invention. The embodiments were chosen and described in order to explain the principles of the present invention and its practical application to enable one skilled in the art to utilize the present invention in various embodiments, and with various modifications, as are suited to the particular use contemplated.

1. A system for augmenting tissue comprising:
needle assembly having:
a hub and a needle, the hub having a first end for engaging a syringe and a second end for engaging the needle;
the needle comprising a hollow shaft with a first end and a second end, each end having an opening respectively into an interior volume of the hollow shaft, the first end engaging the second end of the hub and a second end having a lancet;
the hollow shaft having an outer surface and an inner surface, with a thickness of material there between, the outer surface having a nominal diameter of about 0.353 mm to about 0.367 mm and the inner surface having a nominal diameter of about 0.198 mm to about 0.244 mm, with the nominal thickness of material being about 0.062 to about 0.078 mm, and
a tissue augmentation material;
wherein the tissue augmentation material is capable of passing through the needle.

2. The system of claim 1, further comprising a syringe.

3. The system of claim 1, wherein the tissue augmentation material is stored in the syringe prior to injection through the needle.

4. The system of claim 1, wherein the tissue augmentation material comprises particles of about 15 microns to about 65 microns.

5. The system of claim 4, wherein the tissue augmentation material comprises particles of about 15 microns to about 55 microns.

6. The system of claim 4, wherein the tissue augmentation material comprises particles of about 25 microns to about 45 microns.

7. The system of claim 4, wherein concentration of the particles ranges from about 30% to about 45%.

8. A kit for augmenting tissue comprising:
a needle assembly comprising a hub and a needle, the hub engaging the needle;
the needle comprising a hollow shaft with a first end and a second end, each end having an opening respectively into an interior volume of the hollow shaft, the first end engaging the second end of the hub and a second end having a pointed tip;
the hollow shaft having an outer diameter and an inner diameter, with a thickness of material there between, the nominal outer diameter being about 0.353 mm to about 0.367 mm and the inner surface having a nominal diameter of about 0.198 mm to about 0.244 mm, with the nominal thickness of material being about 0.062 to about 0.078 mm;
an enclosure containing a tissue augmentation material, the tissue augmentation material storable in the enclosure and injectable into a tissue to be augmented through the needle.

9. The kit of claim 8, wherein the enclosure is a syringe, the syringe engageable with a first end of the hub and a second end of the hub engageable with the needle.

10. The kit of claim 8, further comprising a syringe engageable with a first end of the hub and a second end of the hub engageable with the needle.

11. The kit of claim 10, further comprising a plurality of syringes.

12. The kit of claim 8, wherein each of the needles in the plurality of needle assemblies has a different nominal inner diameter and a nominal outer diameter, providing a range of needle sizes for use.

13. The kit of claim 8, wherein the enclosure comprises a plurality of containers.

14. The kit of claim 8, further comprising a plurality of needle assemblies.

15. The kit of claim 8, wherein the needle has a nominal outer diameter of about 0.353 mm and a nominal inner diameter of about 0.198 mm.

16. The kit of claim 8, wherein the needle has a nominal outer diameter of about 0.305 mm and a nominal inner diameter of about 0.198 mm.

17. The kit of claim 8, wherein the needle has a nominal outer diameter of about 0.406 mm and a nominal inner diameter of about 0.254 mm.

18. A system for augmenting tissue comprising:
a syringe;
a needle assembly having:
a hub and a needle, the hub having a first end for engaging the syringe and a second end for engaging the needle;
the needle comprising a hollow shaft with a first end and a second end, each end having an opening respectively into an interior volume of the hollow shaft, the first end engaging the second end of the hub and a second end having a lancet;
the hollow shaft having an outer diameter and an inner diameter, with a thickness of material there between, the nominal outer diameter being about 0.367 mm and the nominal inner diameter of about 0.244 mm, with the nominal thickness of material being about 0.078 mm.
a plurality of injectable particles suspended in a carrier and disposed in the syringe, the plurality of particles having a size distribution within the range of 15 microns to about 65 microns.

19. The system of claim 18, wherein the tissue augmentation material comprises particles of about 15 microns to about 65 microns.

20. The system of claim 18, wherein concentration of the particles ranges from about 30% to about 45%.
